(19) United States
(12) Patent Application Publication
(10) Pub. No.: US 2019/0134249 A1
(43) Pub. Date: May 9, 2019

(54) Method and Apparatus for Rapid Sterilization of Building Air

(71) Applicants: John Taboada, San Antonio, TX (US); John Martin Taboada, San Antonio, TX (US)

(72) Inventors: John Taboada, San Antonio, TX (US); John Martin Taboada, San Antonio, TX (US)

(21) Appl. No.: 15/992,168

(22) Filed: May 29, 2018

Related U.S. Application Data
(63) Continuation-in-part of application No. 15/805,110, filed on Nov. 6, 2017, now Pat. No. 9,981,053.

(51) Int. Cl. A61L 9/20

(52) U.S. Cl. CPC A61L 9/20; A61L 2209/15; A61L 2209/12

(57) ABSTRACT

Methods and systems for rapid sterilization of building air are disclosed, including using a UV source to generate a UV light within an interior volume of duct work of an air handling system; and absorbing the UV light to sterilize. Other embodiments are described and claimed.

### I. CROSS REFERENCE TO RELATED APPLICATIONS

The application is a continuation-in-part application of U.S. Pat. No. 9,981,053, titled “Method and Apparatus for Rapid Sterilization of a Room”, issued May 29, 2018, the contents of which is hereby incorporated by reference, which is a continuation-in-part application of U.S. Pat. No. 9,808,546, titled “Method and Apparatus for Rapid Sterilization of a Room”, issued Nov. 7, 2017, the contents of which is hereby incorporated by reference, which is a continuation-in-part application of U.S. Pat. No. 9,623,131, titled “Method and Apparatus for Rapid Sterilization of Hazmat Suits, Surgical Instruments and the Like”, issued Apr. 18, 2017, the contents of which is hereby incorporated by reference, which claims the benefit of the filing date of U.S. Provisional Patent Application Ser. No. 62/072,306, filed on Oct. 29, 2014, entitled “Method and Apparatus for Rapid Sterilization of Hazmat Suits, Surgical Instruments and the Like,” the entire disclosure of which is hereby incorporated by reference into the present disclosure.

### II. BACKGROUND

There is recurrent need for inactivation of airborne microbes such as H1N1 influenza viruses and other harmful bacteria and pathogens. What is proposed is a rapid, highly efficient means for the inactivation of airborne microbes. An embodiment of the invention is illustrated in the appended figures.

### III. SUMMARY

In one respect, disclosed is an apparatus for sterilizing a room comprising: a laser configured to emit a pulsed laser beam; and a robotic arm configured to direct the pulsed laser beam to a scattering optical element and to change the position of the scattering optical element; wherein the scattering optical element is configured to substantially isotropically scatter the radiation of the pulsed laser beam to sterilize the room.

In another respect, disclosed is a method for sterilizing a room comprising: using a laser to generate a pulsed laser beam; using a robotic arm to direct the pulsed laser beam to a scattering optical element and to change the position of the scattering optical element; and using the scattering optical element to substantially isotropically scatter the radiation of the pulsed laser beam to sterilize the room.

Numerous additional embodiments are also possible.

### IV. BRIEF DESCRIPTION OF THE DRAWINGS

Other objects and advantages of the invention may become apparent upon reading the detailed description and upon reference to the accompanying drawings.

FIG. 1 is a schematic diagram illustrating a rapid sterilizer, in accordance with some embodiments.

FIG. 2 is a schematic diagram illustrating a rapid sterilizer, in accordance with some embodiments.

FIG. 3 is a schematic diagram illustrating a rapid sterilizer, in accordance with some embodiments.

FIG. 4 is a schematic diagram illustrating a rapid sterilizer, in accordance with some embodiments.

FIG. 5 is a schematic diagram illustrating a rapid sterilizer, in accordance with some embodiments.

While the invention is subject to various modifications and alternative forms, specific embodiments thereof are shown by way of example in the drawings and the accompanying detailed description. It should be understood, however, that the drawings and detailed description are not intended to limit the invention to the particular embodiments. This disclosure is instead intended to cover all modifications, equivalents, and alternatives falling within the scope of the present invention as defined by the appended claims.

### V. DETAILED DESCRIPTION

One or more embodiments of the invention are described below. It should be noted that these and any other embodiments are exemplary and are intended to be illustrative of the invention rather than limiting. While the invention is widely applicable to different types of systems, it is impossible to include all of the possible embodiments and contexts of the invention in this disclosure. Upon reading this disclosure, many alternative embodiments of the present invention will be apparent to persons of ordinary skill in the art.

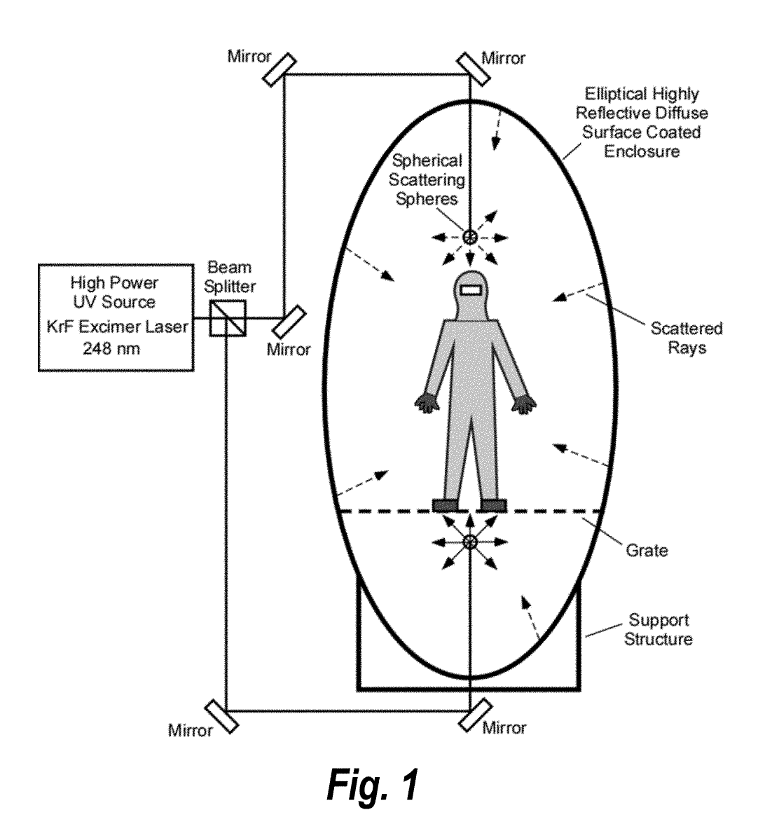

Referring to FIG. 1, an embodiment has a high power Excimer laser using KrF pump media emitting nanosecond UV light pulses at 248 nm. This pulsed UV light has very efficient antibiotic characteristics. The beam from this laser source is split into two beams by a beam splitter (BS) shown in FIG. 1. These beams are steered by means of mirrors (M) to two substantially isotropically scattering optical element, such as spherically scattering spheres, located at the foci of an ellipsoidal highly reflecting diffuse surface coated enclosure. Because the UV light pulses are brought into spherically scattering sources at the foci of an ellipsoidal cavity, the entire cavity will be uniformly illuminated with the UV light pulses. A person (as shown in FIG. 1) will be instantly uniformly illuminated and the hazmat suit they are wearing will be rapidly sterilized and sanitized. To achieve proper centering of the person, a grate with wide open spacing is used as the floor in the enclosure. A support structure holds the enclosure with the major axis vertically. In other embodiments, the laser comprises a fourth harmonic q-switched Nd:YAG solid state laser, a fourth harmonic mode locked Nd:YAG solid state laser, a fourth harmonic q-switched Nd:YLF solid state laser, a fourth harmonic mode locked Nd:YLF solid state laser, a fourth harmonic q-switched Nd:YVO₄ solid state laser, and/or a fourth harmonic mode locked Nd:YVO₄ solid state laser. All of these laser sources are capable of generating short pulses of far UV light which maximizes the lethality of the radiation acting on the pathogenic organisms.

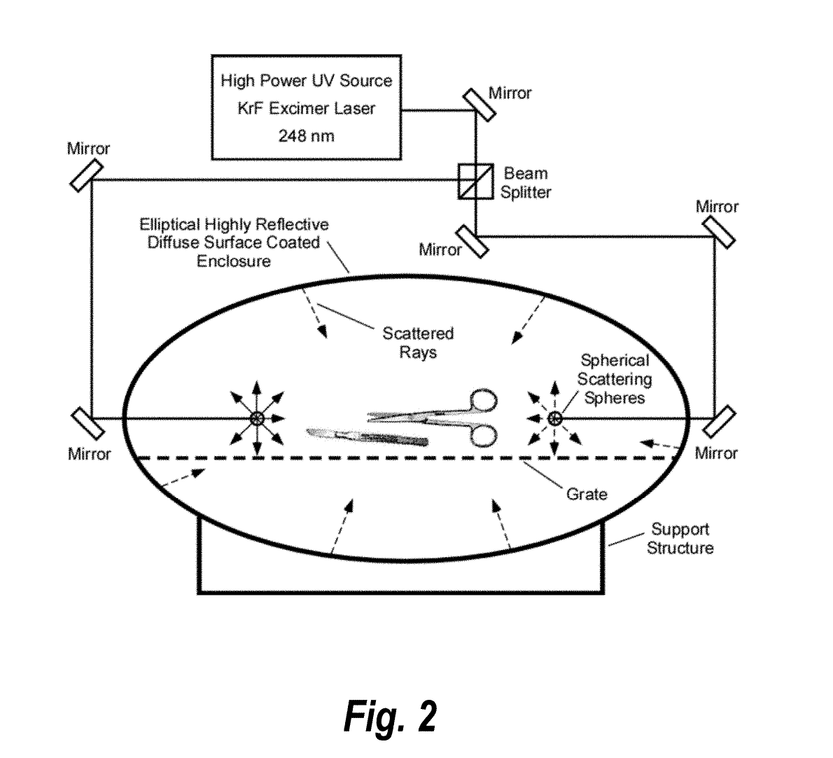

A related embodiment is shown in FIG. 2. In this apparatus, a scaled down version of the above described system is shown. Again a high power KrF Excimer laser emitting nanosecond laser pulses at 248 nm is beam steered to two spherically scattering spheres located at the foci of a properly coated ellipsoidal enclosure as shown in FIG. 2. To be rapidly sterilized and sanitized, the surgical instruments, such as scalpels, forceps, and even endoscopes, are placed on a grate positioned near the major axis of the enclosure. In this arrangement, as in the above disclosed one, the sterilizing pulsed UV light uniformly illuminates the targeted objects placed near the major axis. A support structure as shown keeps the ellipsoidal enclosure horizontal.

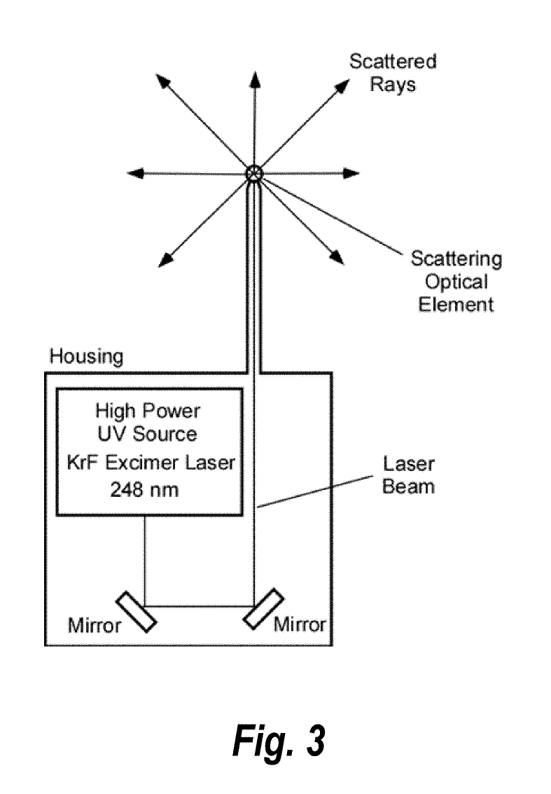

In yet another related embodiment, shown in FIG. 3, the apparatus comprises a scattering optical element configured to scatter incident laser radiation external to a housing. A high power KrF Excimer laser emitting nanosecond laser pulses at 248 nm is beam steered with mirrors to a scattering optical element. Depending on the exact laser source, the wavelength may range from about 200 nm to about 320 nm and the pulsed laser beam may comprise nanosecond or picosecond light pulses. In some embodiments, the scattering optical element is a spherically scattering sphere which isotropically illuminates a room with sterilizing pulsed UV light to rapidly sterilize the room. The spherically scattering sphere comprises a hollow fused silica bulb filled with either solid or hollow fused silica spheres. In some embodiments, the fused silica bulb may be rotated. In an alternate embodiment, the scattering optical element comprises a fiber optic bundle, i.e., a fused bundle of individual fiber optic filaments. In this embodiment, the pulsed UV light is projected radially outward from the fiber optic bundle forming an omnidirectional, substantially planar sheet of UV light and in order to sterilize the entire room, the fiber optic bundle is rotated in a plane substantially orthogonal to the incident laser beam. An example of one such fiber optic bundle was disclosed in U.S. Pat. No. 5,898,809 issued to J. Taboada, et al., which is herein incorporated by reference.

An example embodiment of a spherically scattering sphere may be produced by filling a small fused silica hollow bulb with fused silica solid spheres of an appropriate dimension. The fused silica solid spheres scatter the incident laser radiation in a substantially isotropic manner.

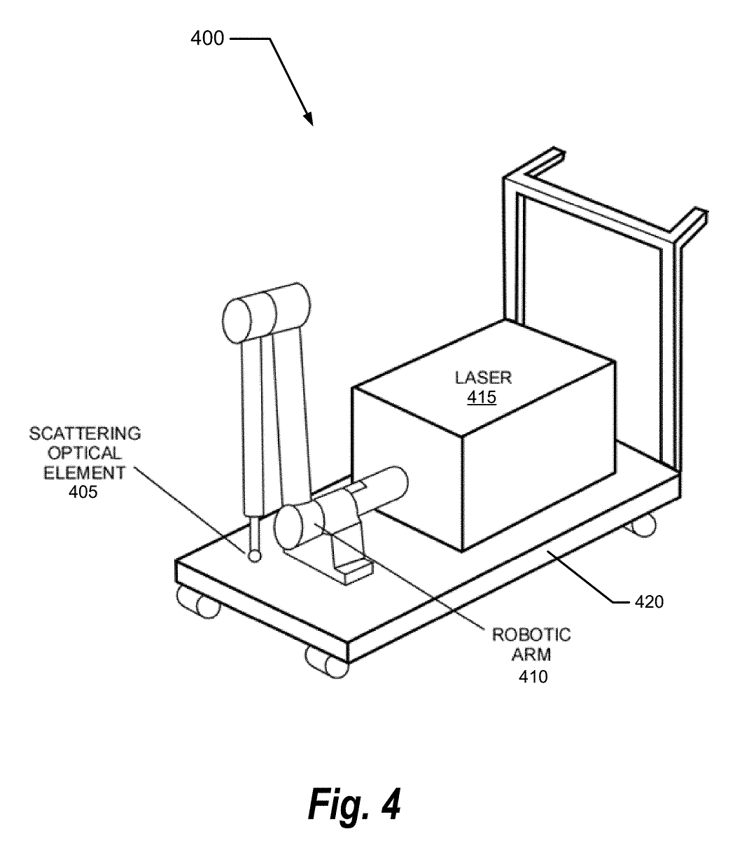

In yet another related embodiment, shown in FIG. 4, the apparatus 400 comprises a scattering optical element 405 positioned at the end of a robotic arm 410 wherein the scattering optical element is configured to scatter incident laser radiation from a laser 415. The robotic arm is designed to move the scattering optical element to different positions in order to be able to get above, below, or behind obstructions or objects in a room that may prevent the thorough sterilization of a room. The robotic arm may have two or more degrees of freedom. One such laser is a high power KrF Excimer laser emitting nanosecond laser pulses at 248 nm. Depending on the exact laser source, the wavelength may range from about 200 nm to about 320 nm and the pulsed laser beam may comprise nanosecond or picosecond light pulses. In some embodiments, the scattering optical element is a spherically scattering sphere which isotropically illuminates a room with sterilizing pulsed UV light to rapidly sterilize the room. The spherically scattering sphere comprises a hollow fused silica bulb filled with either solid or hollow fused silica spheres. In some embodiments, the fused silica bulb may be rotated. In an alternate embodiment, the scattering optical element comprises a fiber optic bundle, i.e., a fused bundle of individual fiber optic filaments. In this embodiment, the pulsed UV light is projected radially outward from the fiber optic bundle forming an omnidirectional, substantially planar sheet of UV light and in order to sterilize the entire room, the fiber optic bundle is rotated in a plane substantially orthogonal to the incident laser beam. An example of one such fiber optic bundle was disclosed in U.S. Pat. No. 5,898,809 issued to J. Taboada, et al., which is herein incorporated by reference.

In some embodiment, the apparatus 400 is mounted onto a movable platform 420. In such an embodiment, the sterilizing laser system may be transported to different areas and rooms that are in need of sterilization.

An example embodiment of a spherically scattering sphere may be produced by filling a small fused silica hollow bulb with fused silica solid spheres of an appropriate dimension. The fused silica solid spheres scatter the incident laser radiation in a substantially isotropic manner.

In alternate embodiments, other UV sterilization light sources may be used, such as placing xenon or mercury vapor lamps at the end of the robotic arm.

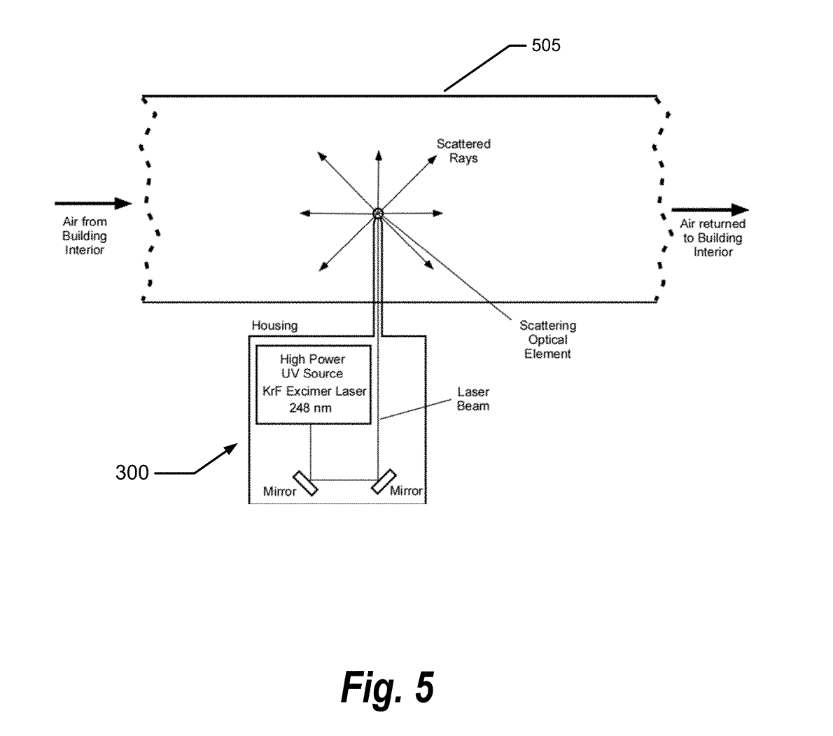

In yet another related embodiment, shown in FIG. 5, the apparatus 300 is integrated into duct work 505 of the air handling system of a building/home. The air handling system, such as a central air conditioning/heating system, draws air from the interior of the building/home, through the furnace, and then back into the interior of the building/home. A high power KrF Excimer laser emitting nanosecond laser pulses at 248 nm is beam steered with mirrors to a scattering optical element. Depending on the exact laser source, the wavelength may range from about 200 nm to about 320 nm and the pulsed laser beam may comprise nanosecond or picosecond light pulses. In some embodiments, the scattering optical element is a spherically scattering sphere which isotropically illuminates the interior of the duct work with sterilizing pulsed UV light to rapidly drawn air. The spherically scattering sphere comprises a hollow fused silica bulb filled with either solid or hollow fused silica spheres. In some embodiments, the fused silica bulb may be rotated. In an alternate embodiment, the scattering optical element comprises a fiber optic bundle, i.e., a fused bundle of individual fiber optic filaments. In this embodiment, the pulsed UV light is projected radially outward from the fiber optic bundle forming an omnidirectional, substantially planar sheet of UV light in order to sterilize the interior of the duct work. An example of one such fiber optic bundle was disclosed in U.S. Pat. No. 5,898,809 issued to J. Taboada, et al., which is herein incorporated by reference.

An example embodiment of a spherically scattering sphere may be produced by filling a small fused silica hollow bulb with fused silica solid spheres of an appropriate dimension. The fused silica solid spheres scatter the incident laser radiation in a substantially isotropic manner.

In alternative embodiment, one or more far-UVC lamps instead of the Excimer laser are configured to illuminate the interior of the duct work 505. The one or more far-UVC lamps may comprise Excimer lamps krypton-bromine at 207 nm and/or krypton-chlorine at 222 nm for example. The one or more Excimer lamps are effective at doses of about 2 mJ/cm² and above.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

The benefits and advantages that may be provided by the present invention have been described above with regard to specific embodiments. These benefits and advantages, and any elements or limitations that may cause them to occur or to become more pronounced are not to be construed as critical, required, or essential features of any or all of the claims. As used herein, the terms “comprises,” “comprising,” or any other variations thereof, are intended to be interpreted as non-exclusively including the elements or limitations which follow those terms. Accordingly, a system, method, or other embodiment that comprises a set of elements is not limited to only those elements, and may include other elements not expressly listed or inherent to the claimed embodiment.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed within the following claims.

1. An apparatus for sterilizing air comprising:
a UV source configured to illuminate an interior volume of duct work of an air handling system.

2. The apparatus of claim 1, wherein the UV source comprises a far-UVC lamp.

3. The apparatus of claim 2, wherein the UV source comprises a wavelength ranging between about 200 nm to about 230 nm.

4. The apparatus of claim 1, wherein the UV source comprises a pulsed laser beam emitted from a laser outside of the enclosure.

5. The apparatus of claim 4, wherein the laser comprises at least one of a KrF Excimer laser, a fourth harmonic q-switched Nd:YAG solid state laser, a fourth harmonic mode locked Nd:YAG solid state laser, a fourth harmonic q-switched Nd:YLF solid state laser, a fourth harmonic mode locked Nd:YLF solid state laser, a fourth harmonic q-switched Nd:YVO₄ solid state laser, and a fourth harmonic mode locked Nd:YVO₄ solid state laser.

6. The apparatus of claim 4, further comprising a scattering optical element configured to intercept and substantially isotropically scatter the radiation of the pulsed laser beam.

7. The apparatus of claim 4, wherein the pulsed laser beam comprises a wavelength ranging between about 200 nm to about 320 nm.

8. The apparatus of claim 4, wherein the laser is q-switched and/or mode locked.

9. The apparatus of claim 4, wherein the pulsed laser beam comprises nanosecond or picosecond light pulses.

10. A method for sterilizing comprising:
using a UV source to generate a UV light within an interior volume of duct work of an air handling system; and
absorbing the UV light to sterilize.

11. The method of claim 10, wherein the UV source comprises a far-UVC lamp.

12. The method of claim 11, wherein the UV source comprises a wavelength ranging between about 200 nm to about 230 nm.

13. The method of claim 10, wherein the UV source comprises a pulsed laser beam emitted from a laser outside of the enclosure.

14. The method of claim 13, wherein the laser comprises at least one of a KrF Excimer laser, a fourth harmonic q-switched Nd:YAG solid state laser, a fourth harmonic mode locked Nd:YAG solid state laser, a fourth harmonic q-switched Nd:YLF solid state laser, a fourth harmonic mode locked Nd:YLF solid state laser, a fourth harmonic q-switched Nd:YVO₄ solid state laser, and a fourth harmonic mode locked Nd:YVO₄ solid state laser.

15. The method of claim 13, further comprising a scattering optical element configured to intercept and substantially isotropically scatter the radiation of the pulsed laser beam.

16. The method of claim 13, wherein the pulsed laser beam comprises a wavelength ranging between about 200 nm to about 320 nm.

17. The method of claim 13, wherein the laser is q-switched and/or mode locked.

18. The method of claim 13, wherein the pulsed laser beam comprises nanosecond or picosecond light pulses.
